(19) United States
(12) Patent Application Publication
(10) Pub. No.: US 2008/0233632 A1
(43) Pub. Date: Sep. 25, 2008

(54) Process for Labeling and Purification of Nucleic Acids of Interest Present in a Biological Sample to be Treated in a Single Reaction Vessel

(75) Inventors: Ali Laayoun, Toussieu (FR); Lionel Menou, Saint-Genis-Laval (FR); Frederic Ginot, Saint-Egreve (FR)

Correspondence Address: OLIFF & BERRIDGE, PLC, P.O. BOX 320850, ALEXANDRIA, VA, 22320-4850, US

(73) Assignee: BIOMERIEUX, Marcy L'Etoile (FR)

(21) Appl. No.: 11/658,028

(22) PCT Filed: Jul. 21, 2005
(86) PCT No.: PCT/FR2005/050601

§ 371 Date: Jan. 22, 2007

(30) Foreign Application Priority Data
Jul. 23, 2004 (FR) 0451632

(51) Int. Cl. C07H 1/00

(52) U.S. Cl. 435/270; 536/25.32

(57) ABSTRACT

The present invention relates to a process for labeling and purification of nucleic acids of interest present in a biological sample to be treated, comprising:
taking a single reaction vessel,
introducing into the reaction vessel:
   the biological sample,
   at least one labeling reagent for nucleic acids,
   at least one solid support enabling the adsorption of said nucleic acids,
   any ingredient necessary for the labeling of the nucleic acids and/or for the immobilization of said nucleic acids on the support,
incubating the contents of the reaction vessel, and
isolating the nucleic acids thus labeled.

The invention finds a preferred application in the diagnostics field.

The present invention relates to a process for labeling of nucleic acids in the presence of at least one solid support.

The state of the technology shows that many methods exist for labeling nucleotides, oligonucleotides or nucleic acids.

A first method consists in attaching the label to the base, whether the latter is natural or modified. A second method proposes attaching the label to the sugar, here also whether it is natural or modified. The object of a third method is the attachment of the label to the phosphate.

Labeling on the base has in particular been used in the approach of nucleic acid labeling by incorporation of directly labeled nucleotides.

Labeling on the sugar is often used in the case of nucleic probes prepared by chemical synthesis.

Labeling on the phosphate has also been used to introduce functionalized arms and labels during the chemical synthesis of oligonucleotides.

In fact, the skilled person who has to perform the labeling of a nucleotide, or of a nucleotide analog or of a nucleic acid, is inclined to perform this attachment onto the base or onto the sugar which offer him greater convenience and more alternatives. Moreover, this is what emerges from the study of many documents, such as EP-A-0.329.198, EP-A-0.302.175, EP-A-0.097.373, EP-A-0.063.879, U.S. Pat. No. 5,449,767, U.S. Pat. No. 5,328,824, WO-A-93/16094, DE-A-3.910.151, EP-A-0.567.841 for the base or EP-A-0.286.898 for the sugar.

The attachment of the label onto the phosphate is a more complex technique than the technique consisting in functionalizing the base or the sugar and has been much less used in particular on account of the low reactivity of the phosphate (see for example Jencks W. P. et al., J. Amer. Chem. Soc., 82, 1778-1785, 1960). Likewise, in the review by O'Donnel and McLaughlin (“Reporter groups for the analysis of nucleic acid structure”, p 216-243, in “Bioorganic Chemistry: Nucleic Acids”, Ed Hecht S. M., Oxford University Press, 1996) relating to methods for the introduction of probes into oligonucleotide fragments, the efficient alkylation of the internucleotide phosphodiester is considered to be impossible.

The patent application WO-A-99/65926 describes a process for labeling of a synthetic or natural ribonucleic acid (RNA) which consists in fragmenting the RNA and labeling at the terminal phosphate. This document describes a certain number of functional groups which can be used for labeling in combination with fragmentation such as the hydroxyl, amine, hydrazine, alkoxyamine, alkyl halide, benzylic type alkyl halide groups and in particular the 5-(bromomethyl)fluorescein derivative. These functional groups make it possible to label the nucleic acids, but a fragmentation step has to be included in order to have efficient labeling, since this labeling takes place on the phosphate liberated during the fragmentation. Moreover, it is necessary to add a considerable excess of labeling reagent relative to the RNA in order to obtain efficient labeling, which gives rise to problems of background noise generated by the excess label. Finally, this method does not work efficiently on double-strand DNA.

Novel reagents still more efficient in terms of the labeling yield have appeared. These are specific as regards the labeling position and in particular they do not affect the hybridization properties of the bases involved in the formation of the double helix, via hydrogen bonds, which are utilizable both for DNA and RNA, and finally which make it possible to label nucleotides, oligonucleotides and nucleic acids, whether natural or prepared by enzymatic amplification, equally well.

The Applicant has already proposed such novel labels which meet the aforesaid conditions and which use the diazomethyl group as the reactive group for the labeling. This is for example the case in:
patent application WO-A-02/090319,
patent application WO-A-02/090584,
a not yet published application of 26 Mar. 2004 filed in France under the number: FR04/50600 and entitled: “Réactifs de marquage, procédés de synthese de tels réactifs et procédés de detection de molecules biologiques” or again
the article by Laayoun et al. published in Bioconjugate Chem. 2003, 14, 1298-1306 and entitled: “Aryldiazomethanes for Universal Labeling of Nucleic Acids and Analysis on DNA Chips”, to which the reader may refer in order better to understand the methods for synthesis and utilization of such components.
   The content of those documents enumerated above is incorporated here for reference.

The state of the technology also describes the use of a solid support, in particular of silica, and more particularly in the form of a powder, gel or magnetic particles, to purify the nucleic acids before or after labeling, in a process leading to detection by specific hybridization (relating to DNA chips, but also ELOSA plates or types of rapid test). The purification before labeling makes it possible to considerably improve the labeling yield, and post-labeling purification makes it possible to decisively improve the hybridization yield and the signal/noise ratio, as is necessary for good test sensitivity.

This is the case in the patent EP-B-0.389.063 which proposes a process for isolation of nucleic acids containing a complex biological starting material, the process therefore comprising mixing the starting material with a chaotropic substance and a solid phase, the solid phase enabling the attachment of nucleic acids which can subsequently be washed or eluted from the remainder of the complexes thus created.

The content of that patent is likewise incorporated here for reference.

In fact, these two techniques are used independently, the purification and labeling steps being performed in different disposable vessels, with operating methods which are likewise different. The presence of two steps thus necessitates transfers of liquids, potentially contamination factors, loss of material, and, in general moderately automatable.

On the other hand, the process combining labeling and purification on silica presents numerous advantages, which had until now remained unsuspected by the skilled person.

The first advantage is that the process according to the invention in a reaction medium simultaneously allowing the labeling reaction and the capture, for example by adsorption, of the nucleic acid on magnetic beads of silica does not entail any decrease in the efficiency of the labeling, labeling which is thus not affected by the presence of the magnetic silica.

The second advantage is that the process is, comparatively speaking, much faster to perform than a process in two distinct steps, labeling and purification.

The third advantage of the invention is that it makes it possible to concentrate the labeled nucleic acids in a very small volume, of the order of 0.1 to 10 μl, the nucleic acids being attached to the beads, and being capable of elution by a simple standard hybridization buffer without recourse to an elution buffer. It is thus possible completely to eliminate this dilution step and to improve the sensitivity of the process, the nucleic acids being more concentrated during the hybridization.

Moreover, the process is readily automatable, which constitutes a fourth advantage, owing to the flexibility of the use of magnetic beads, and to the relative simplicity of the process (heating, washing, elution). The use of magnetic beads in particular makes it possible to vary the capture capacity of the system very easily, by simple modification of the quantity of beads used. The process can also be used in a system using a continuous flow, making it possible to simplify the washing steps. There is then no pipetting of liquid to perform.

A fifth advantage of this process lies in the fact that it allows the transfer of the nucleic acids in solid form, in other words adsorbed onto magnetic beads of silica, which renders it easily utilizable in microcomponents, a technology which is currently undergoing rapid development.

Finally, a sixth advantage among others, which is also connected with the automation of the process (fourth advantage described) consists in a process which can be completely integrated in a single tube, from the purification of the nucleic acid to the hybridization on chip, passing via the labeling, if, of course, this is in the context of a process not requiring an amplification step. This results in a reduction in the contamination risks and a decrease in the number of disposable vessels used.

The present invention essentially relates to a process for labeling and purification of nucleic acids of interest which are present in a biological sample to be treated, consisting in:
taking a single reaction vessel,
introducing into the reaction vessel:
   the biological sample,
   at least one reagent for labeling nucleic acids,
   at least one solid support enabling the adsorption of said nucleic acids,
   any ingredient necessary for the labeling of the nucleic acids and/or for the immobilization of said nucleic acids on the support,
incubating the contents of the reaction vessel, and
isolating the nucleic acids thus labeled.

According to a first implementation mode of the invention, the above labeling and purification process is characterized in that the nucleic acids treated can consist of single-strand and/or double-strand, synthetic and/or natural DNA and/or RNA.

According to a second implementation mode of the invention, the above labeling and purification process is characterized in that the labeling reagent can enable:

the fragmentation of the nucleic acids in a nonspecific manner to generate a plurality of nucleic acid fragments, and

the labeling of a plurality of these fragments on the terminal phosphate situated at the 3′ and/or 5′ end, said terminal phosphate having been liberated during the fragmentation.

Still according to this second implementation mode of the invention, the labeling of the 3′ or 5′ end of a nucleic acid fragment can be performed by attachment of a reactive group borne by a label to the phosphate in the 2′ position, 3′ position or cyclic 2′-3′-monophosphate position, with reference to the ribose.

Again according to this second implementation mode of the invention, the fragmentation and/or the labeling of the 3′ or 5′ end of a nucleic acid fragment can be performed by attachment of a nucleophilic, electrophilic or halogen group borne by a label to the phosphate in the 2′ position, 3′ or cyclic 2′-3′-monophosphate position, with reference to the ribose.

According to the options for implementation of this second implementation mode of the invention, the fragmentation of the nucleic acids can be effected by:
enzymatic means (nucleases),
chemical means (metal cations, such as Mg⁺⁺, Mn⁺⁺, Cu⁺⁺, Co⁺⁺ and/or Zn⁺⁺ ions, whether or not in combination with a chemical catalyst, for example N-methyl-imidazole, or any chemical molecule having an affinity for RNA and bearing an imidazole nucleus or a substituted analog) or
physical means (by sonication or by radiation).

Whatever the implementation mode, the labeling of the 3′ or 5′ end of a fragment of RNA can be performed by attachment of a molecule R—X, where R consists of the label and X is the bonding agent between the label and the RNA, such as a hydroxyl, amine, hydrazine, alkoxylamine, alkyl halide, phenyl-methyl halide, iodoacetamide or maleimide group, to the phosphate bound at the 2′ position, 3′ position or cyclic 2′-3′-monophosphate position of the ribose.

Whatever the implementation mode, the labeling on the phosphate group can be effected by means of 5-(bromomethyl)-fluorescein.

According to a particular implementation mode, the labeling and purification process according to the invention, is characterized in that the labeling reagent can be contacted with the nucleic acids in homogenous solution, in an essentially aqueous buffer, said labeling reagent being stable to heat and of formula (0):

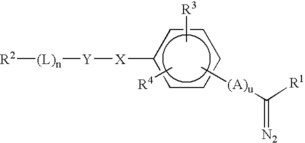

wherein:
R¹ represents H or an alkyl, aryl or substituted aryl group,
R² represents a detectable label or at least two detectable labels linked together by at least one multimeric structure,
L is a linking arm comprising a linear chain of at least two covalent bonds and n a whole number equal to 0 or 1,
R³ and R⁴ independently of one another represent: H, NO₂, Cl, Br, F, I, R²-(L)ₙ-Y—X—, OR, SR, NR₂, R, NHCOR, CONHR or COOR with R=alkyl or aryl,
A is a linking arm comprising at least one covalent double bond enabling the conjugation of the diazo group with the aromatic ring and u is a whole number lying between 0 and 2, preferably 0 or 1, and
—Y—X— represents —CONH—, —NHCO—, —CH₂O— or —CH₂S—.

According to this particular implementation mode, the labeling reagent can be of formula (1):

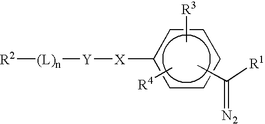

wherein:
R¹ represents H or an alkyl, aryl or substituted aryl group,
R² represents a detectable label or at least two detectable labels linked together by at least one multimeric structure,
L is a linking arm comprising a linear chain of at least two covalent bonds and n a whole number equal to 0 or 1,
R³ and R⁴ independently of one another represent: H, NO₂, Cl, Br, F, I, R²-(L)ₙ-Y—X—, OR, SR, NR₂, R, NHCOR, CONHR or COOR with R=alkyl or aryl, and
—Y—X— represents —CONH—, —NHCO—, —CH₂O— or —CH₂S—.

According to the two preceding characteristics, the reagent can be of formula (2):

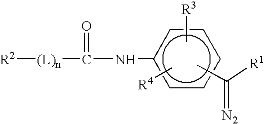

wherein:
R¹ represents H or an alkyl, aryl or substituted aryl group,
R² represents a detectable label or at least two detectable labels linked together by at least one multimeric structure,
L is a linking arm comprising a linear chain of at least two covalent bonds and n a whole number equal to 0 or 1, and
R³ and R⁴ independently of one another represent: H, NO₂, Cl, Br, F, I, R²-(L)ₙ-Y—X—, OR, SR, NR₂, R, NHCOR, CONHR or COOR with R=alkyl or aryl.

According to a first version of the two previous implementation modes, the reagent can be of formula (3):

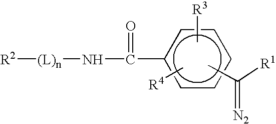

wherein:
R¹ represents H or an alkyl, aryl or substituted aryl group,
R² represents a detectable label or at least two detectable labels linked together by at least one multimeric structure,
L is a linking arm comprising a linear chain of at least two covalent bonds and n a whole number equal to 0 or 1, and
R³ and R⁴ independently of one another represent: H, NO₂, Cl, Br, F, I, R²-(L)ₙ-Y—X—, OR, SR, NR₂, R, NHCOR, CONHR or COOR with R=alkyl or aryl.

According to a second version of the two preceding implementation modes, the reagent can be of formula (4):

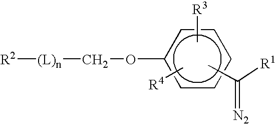

wherein:
R¹ represents H or an alkyl, aryl or substituted aryl group,
R² represents a detectable label or at least two detectable labels linked together by at least one multimeric structure,
L is a linking arm comprising a linear chain of at least two covalent bonds and n a whole number equal to 0 or 1, and
R³ and R⁴ independently of one another represent: H, NO₂, Cl, Br, F, I, R²-(L)ₙ-Y—X—, OR, SR, NR₂, R, NHCOR, CONHR or COOR with R=alkyl or aryl.

According to another version of the first of these two preceding implementation modes, the reagent can be of formula (21):

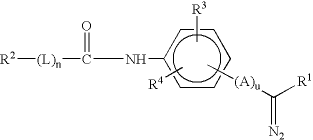

wherein:
R¹ represents H or an alkyl, aryl or substituted aryl group,
R² represents a detectable label or at least two detectable labels linked together by at least one multimeric structure,
L is a linking arm comprising a linear chain of at least two covalent bonds and n a whole number equal to 0 or 1,
A is a linking arm comprising at least one covalent double bond enabling the conjugation of the diazo group with the aromatic ring and u equals 1, and
R³ and R⁴ independently of one another represent: H, NO₂, Cl, Br, F, I, R²-(L)ₙ-Y—X—, OR, SR, NR₂, R, NHCOR, CONHR or COOR with R=alkyl or aryl.

According to yet another version of the first of these two preceding implementation modes, the reagent can be of formula (22):

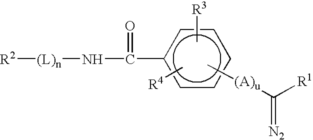

wherein:
R¹ represents H or an alkyl, aryl or substituted aryl group,
R² represents a detectable label or at least two detectable labels linked together by at least one multimeric structure,
L is a linking arm comprising a linear chain of at least two covalent bonds and n a whole number equal to 0 or 1,
A is a linking arm comprising at least one covalent double bond enabling the conjugation of the diazo group with the aromatic ring and u equals 1, and
R³ and R⁴ independently of one another represent: H, NO₂, Cl, Br, F, I, R²-(L)ₙ-Y—X—, OR, SR, NR₂, R, NHCOR, CONHR or COOR with R=alkyl or aryl.

According to still another version of the first of these two preceding implementation modes, the reagent can be of formula (23):

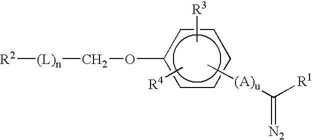

wherein:
R¹ represents H or an alkyl, aryl or substituted aryl group,
R² represents a detectable label or at least two detectable labels linked together by at least one multimeric structure,
L is a linking arm comprising a linear chain of at least two covalent bonds and n a whole number equal to 0 or 1,
A is a linking arm comprising at least one covalent double bond enabling the conjugation of the diazo group with the aromatic ring and u equals 1, and
R³ and R⁴ independently of one another represent: H, NO₂, Cl, Br, F, I, R²-(L)ₙ-Y—X—, OR, SR, NR₂, R, NHCOR, CONHR or COOR with R=alkyl or aryl.

According to all the versions of these two preceding implementation modes, the reagent, R³ and R⁴ can independently of each other represent: H, NO₂, OCH₃, —CO—NH—(CH₂)₃—(O—CH₂—CH₂)₃—CH₂—NH—R² or —CO—NH—(CH₂)₃—(O—CH₂—CH₂)₄—CH₂—NH—R².

According to another version of these preceding implementation modes, the reagent can be of formula (7):

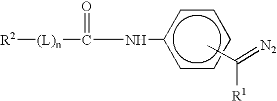

wherein:
R¹ represents H or an alkyl, aryl or substituted aryl group,
R² represents a detectable label or at least two detectable labels linked together by at least one multimeric structure, and
L is a linking arm comprising a linear chain of at least two covalent bonds and n a whole number equal to 0 or 1.

According to yet another version of these preceding implementation modes, the reagent can be of formula (24):

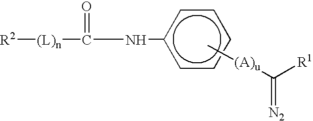

wherein:
R¹ represents H or an alkyl, aryl or substituted aryl group,
R² represents a detectable label or at least two detectable labels linked together by at least one multimeric structure, and
A is a linking arm comprising at least one covalent double bond enabling the conjugation of the diazo group with the aromatic ring and u equals 1.

Whatever the version of these preceding implementation modes, the structure R²-(L)ₙ- of the reagent can be of formula (5):

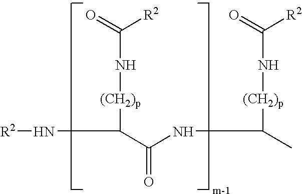

wherein:
R² represents a detectable label,
m is a whole number lying between 1 and 100, and
p is a whole number lying between 1 and 10.

Whatever the version of these preceding implementation modes, the reagent can be of formula (6):

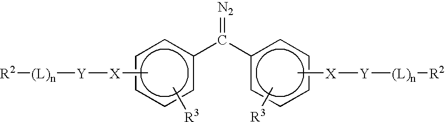

wherein:
R² represents a detectable label or at least two detectable labels linked together by at least one multimeric structure,
R³ represents H, NO₂, Cl, Br, F, I, R²-(L)ₙ-Y—X—, OR, SR, NR₂, R, NHCOR, CONHR or COOR with R=alkyl or aryl,
L is a linking arm comprising a linear chain of at least two covalent bonds and n a whole number equal to 0 or 1, and
—Y—X— represents —CONH—, —NHCO—, —CH₂O— or —CH₂S—.

Whatever the version of these preceding implementation modes, the reagent can be of formula (25):

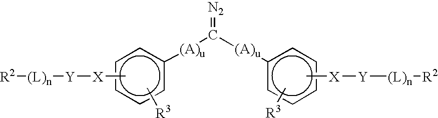

wherein:
R² represents a detectable label or at least two detectable labels linked together by at least one multimeric structure,
R³ represents H, NO₂, Cl, Br, F, I, R²-(L)ₙ-Y—X—, OR, SR, NR₂, R, NHCOR, CONHR or COOR with R=alkyl or aryl,
L is a linking arm comprising a linear chain of at least two covalent bonds and n a whole number equal to 0 or 1,
A is a linking arm comprising at least one covalent double bond enabling the conjugation of the diazo group with the aromatic ring and u equals 1, and
—Y—X— represents —CONH—, —NHCO—, —CH₂O— or —CH₂S—.

Whatever the version of these preceding implementation modes, the reagent can be of formula (14):

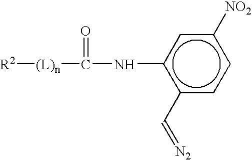

wherein:
R² represents a detectable label or at least two detectable labels linked together by at least one multimeric structure, and
L is a linking arm comprising a linear chain of at least two covalent bonds and n a whole number equal to 0 or 1.

Whatever the version of these preceding implementation modes, the reagent can be of formula (26):

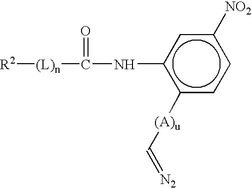

wherein:
R² represents a detectable label or at least two detectable labels linked together by at least one multimeric structure,
A is a linking arm comprising at least one covalent double bond enabling the conjugation of the diazo group with the aromatic ring and u equals 1, and
L is a linking arm comprising a linear chain of at least two covalent bonds and n a whole number equal to 0 or 1.

Whatever the version of these preceding implementation modes, the reagent can be of formula (15):

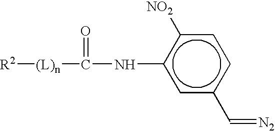

wherein:
R² represents a detectable label or at least two detectable labels linked together by at least one multimeric structure, and
L is a linking arm comprising a linear chain of at least two covalent bonds and n a whole number equal to 0 or 1.

Whatever the version of these preceding implementation modes, the reagent can be of formula (27):

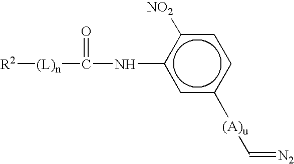

wherein:
R² represents a detectable label or at least two detectable labels linked together by at least one multimeric structure,
A is a linking arm comprising at least one covalent double bond enabling the conjugation of the diazo group with the aromatic ring and u equals 1, and
L is a linking arm comprising a linear chain of at least two covalent bonds and n a whole number equal to 0 or 1.

According to the totality of the versions of these preceding implementation modes, the constituent L of the reagent can contain an —(O—CH₂—CH₂)— moiety, repeated from 1 to 20 times, preferably from 1 to 10 times, and still more preferably from 2 to 5 times.

According to the very first two implementation modes, the labeling reagent can enable the labeling and the fragmentation of a single or double-strand nucleic acid according to the following steps:

fragmenting the nucleic acid,

attaching a label onto at least one of the fragments by means of a labeling reagent selected from compounds of formula (19):

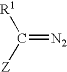

wherein:
R¹ represents H or an alkyl, aryl or substituted aryl group, and
Z contains a detectable label,
   said reagent coupling covalently and mainly onto at least one phosphate of said fragment.

According to the preceding implementation mode, the labeling reagent can be selected from compounds of formula (20):

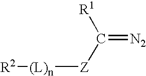

wherein:
R¹ represents H or an alkyl, aryl or substituted aryl group,
R² is a detectable label or at least two detectable labels linked together by at least one multimeric structure,
L is a linking arm comprising a linear chain of at least two covalent bonds and n is equal to 0 or 1, and
Z is selected from:

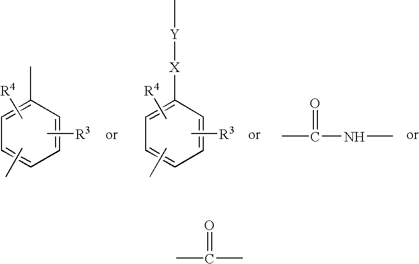

wherein:
   R³ and R⁴ independently of one another represent: H, NO₂, Cl, Br, F, I, R²-(L)ₙ-Y—X—, OR, SR, NR₂, R, NHCOR, CONHR or COOR with R=alkyl or aryl, and
   —Y—X— represents —CONH—, —NHCO—, —CH₂O— or —CH₂S—.

According to one implementation version, group Z can consist of

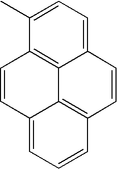

According to another implementation version, the fragmentation and the labeling can be performed in two steps.

According to another implementation version, the fragmentation and the labeling can be performed in one step.

According to another implementation version, the labeling can be performed in homogenous, essentially aqueous solution.

According to another implementation version, the fragmentation can be performed by enzymatic, physical or chemical means.

According to the very first two implementation modes, the labeling reagent can be contacted in homogenous solution, in an essentially aqueous buffer, said labeling reagent being stable to heat and of formula (8):

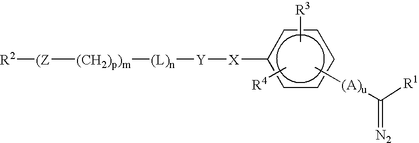

wherein:
R¹ represents H or an alkyl, aryl or substituted aryl group,
R² represents a detectable label or at least two detectable labels linked together by at least one multimeric structure,
L is a linking arm comprising a linear chain of at least two covalent bonds and n a whole number equal to 0 or 1,
R³ and R⁴ independently of one another represent: H, NO₂, Cl, Br, F, I, R²-(L)ₙ-Y—X—, OR, SR, NR₂, R, NHCOR, CONHR, COOR, —CO—NH—(CH₂)₃—(O—CH₂—CH₂)₃—CH₂—NH—R², or —CO—NH—(CH₂)₃—(O—CH₂—CH₂)₄—CH₂—NH—R² with R=alkyl or aryl,
A is a linking arm comprising at least one covalent double bond enabling the conjugation of the diazo group with the aromatic ring and u is a whole number lying between 0 and 2, preferably 0 or 1,
—Y—X— represents —CONH—, —NHCO—, —CH₂O— or —CH₂S—,
-Z- represents —NH—, —NHCO—, —CONH— or —O—,
m is a whole number lying between 1 and 10, preferably between 1 and 3, and
p is a whole number lying between 1 and 10, preferably between 1 and 3.

According to one version of the preceding implementation mode, the reagent can be of formula (9):

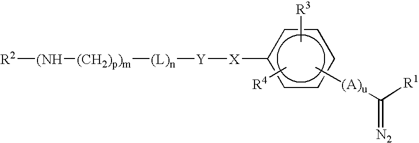

wherein:
R¹ represents H or an alkyl, aryl or substituted aryl group,
R² represents a detectable label or at least two detectable labels linked together by at least one multimeric structure,
L is a linking arm comprising a linear chain of at least two covalent bonds and n a whole number equal to 0 or 1,
R³ and R⁴ independently of one another represent: H, NO₂, Cl, Br, F, I, R²-(L)ₙ-Y—X—, OR, SR, NR₂, R, NHCOR, CONHR, COOR, —CO—NH—(CH₂)₃—(O—CH₂—CH₂)₃—CH₂—NH—R², or —CO—NH—(CH₂)₃—(O—CH₂—CH₂)₄—CH₂—NH—R² with R=alkyl or aryl, and
—Y—X— represents —CONH—, —NHCO—, —CH₂O— or —CH₂S—,
m is a whole number lying between 1 and 10, preferably between 1 and 3, and
p is a whole number lying between 1 and 10, preferably between 1 and 3.

According to the two preceding implementation modes, in the formula of the reagent, p can be less than or equal to m.

According to the three preceding implementation modes, the reagent can be of formula (10):

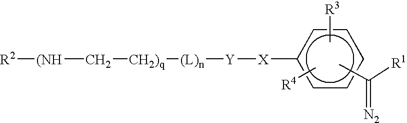

wherein:
R¹ represents H or an alkyl, aryl or substituted aryl group,
R² represents a detectable label or at least two detectable labels linked together by at least one multimeric structure,
L is a linking arm comprising a linear chain of at least two covalent bonds and n a whole number equal to 0 or 1,
R³ and R⁴ independently of one another represent: H, NO₂, Cl, Br, F, I, R²-(L)ₙ-Y—X—, OR, SR, NR₂, R², NHCOR, CONHR, COOR, —CO—NH—(CH₂)₃—(O—CH₂—CH₂)₃—CH₂—NH—R², or —CO—NH—(CH₂)₃—(O—CH₂—CH₂)₄—CH₂—NH—R² with R=alkyl or aryl, and
q is a whole number lying between 1 and 10, preferably between 1 and 3.

According to the four preceding implementation modes, the reagent can be of formula (11):

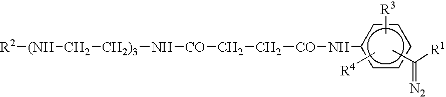

wherein:
R¹ represents H or an alkyl, aryl or substituted aryl group,
R² represents a detectable label or at least two detectable labels linked together by at least one multimeric structure,
L is a linking arm comprising a linear chain of at least two covalent bonds and n a whole number equal to 0 or 1, and
R³ and R⁴ independently of one another represent: H, NO₂, Cl, Br, F, I, R²-(L)ₙ-Y—X—, OR, SR, NR₂, R², NHCOR, CONHR, COOR, —CO—NH—(CH₂)₃—(O—CH₂—CH₂)₃—CH₂—NH—R², or —CO—NH—(CH₂)₃—(O—CH₂—CH₂)₄—CH₂—NH—R² with R=alkyl or aryl.

According to one implementation mode, the reagent, R² can consist of a D-Biotin residue of formula (12):

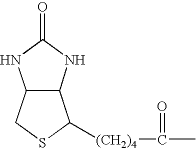

According to the six preceding implementation modes, the reagent, R¹ consists of: CH₃, and R³ and R⁴ each represent: H.

According to the seven preceding implementation modes, the structure -(L)ₙ- of the reagent can consist of:
permine or N,N′-Bis(3-aminopropyl)-1,4-diaminobutane: NH₂—(CH₂)₃—NH—(CH₂)₄—NH—(CH₂)₃—NH₂, or
spermidine or N-(3-aminopropyl)-1,4-butanediamine: H₂N—(CH₂)₄—NH—(CH₂)₃—NH₂, or
a derivative containing an alanine moiety: NH₂—CH₂—CH₂—COOH.

According to the very first two implementation modes, the labeling reagent stable to heat can be contacted in homogenous solution, in an aqueous buffer and is of formula (13):

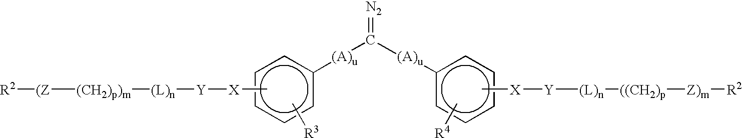

wherein:
R¹ represents H or an alkyl, aryl or substituted aryl group,
R² represents a detectable label or at least two detectable labels linked together by at least one multimeric structure,
L is a linking arm comprising a linear chain of at least two covalent bonds and n a whole number equal to 0 or 1,
R³ and R⁴ independently of one another represent: H, NO₂, Cl, Br, F, I, R²-(L)ₙ-Y—X—, OR, SR, NR₂, R², NHCOR, CONHR, COOR, —CO—NH—(CH₂)₃—(O—CH₂—CH₂)₃—CH₂—NH—R², or —CO—NH—(CH₂)₃—(O—CH₂—CH₂)₄—CH₂—NH—R² with R=alkyl or aryl,
A is a linking arm comprising at least one covalent double bond enabling the conjugation of the diazo group with the aromatic ring and u is a whole number lying between 0 and 2, preferably 0 or 1,
—Y—X— represents —CONH—, —NHCO—, —CH₂O— or —CH₂S—,
-Z- represents —NH—, —NHCO—, —CONH— or —O—,
m is a whole number lying between 1 and 10, preferably between 1 and 3, and
p is a whole number lying between 1 and 10, preferably between 1 and 3.

According to the preceding implementation mode, the reagent can be of formula (16):

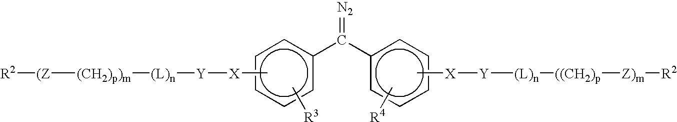

wherein:
R¹ represents H or an alkyl, aryl or substituted aryl group,
R² represents a detectable label or at least two detectable labels linked together by at least one multimeric structure,
L is a linking arm comprising a linear chain of at least two covalent bonds and n a whole number equal to 0 or 1,
R³ and R⁴ independently of one another represent: H, NO₂, Cl, Br, F, I, R²-(L)ₙ-Y—X—, OR, SR, NR₂, R², NHCOR, CONHR, COOR, —CO—NH—(CH₂)₃—(O—CH₂—CH₂)₃—CH₂—NH—R², or —CO—NH—(CH₂)₃—(O—CH₂—CH₂)₄—CH₂—NH—R² with R=alkyl or aryl,
—Y—X— represents —CONH—, —NHCO—, —CH₂O— or —CH₂S—,
-Z- represents —NH—, —NHCO—, —CONH— or —O—,
m is a whole number lying between 1 and 10, preferably between 1 and 3, and
p is a whole number lying between 1 and 10, preferably between 1 and 3.

According to the ten preceding implementation modes, the constituent L of the reagent can contain an —(O—CH₂—CH₂)— moiety, repeated from 1 to 20 times, preferably from 1 to 10 times, and still more preferably from 2 to 5 times, -Z- then being represented by —NH—, —NHCO— or —CONH—.

Whatever the implementation mode, the solid support can consist of particles of silica.

Whatever the implementation mode, the solid support can consist of magnetic particles covered with silica.

According to the two preceding implementation modes, the particles of silica comprising the solid support can have particle sizes lying between 0.1 and 500 μm and preferably between 1 and 200 μm.

Whatever the implementation mode, one of the supplementary ingredients enabling the labeling can consist of an alcohol, preferably Isopropanol.

According to the preceding implementation mode, the isopropanol can comprise 70% v/v of the final mixture.

Whatever the implementation mode, one of the supplementary ingredients enabling the cellular release and hence the adsorption of the nucleic acids onto the solid support can consist of a chaotropic agent.

According to the preceding implementation mode, the chaotropic agent used can be a guanidinium salt, sodium iodide, potassium iodide, sodium (iso)thiocyanate, urea or mixtures of these compounds.

According to the preceding implementation mode, the guanidinium salt used can be guanidinium (iso)thiocyanate.

Whatever the implementation mode, the solid phase-nucleic acid complexes can be separated from the liquids by sedimentation and rejection of the supernatant and then washing of the complexes with a washing buffer containing a chaotropic substance.

According to the preceding implementation mode, the solid phase-nucleic acid complexes, after washing with the washing buffer, can then be washed again with one or several organic solvents, and then subjected to a drying process.

According to the preceding implementation mode, the nucleic acid present in the solid phase-nucleic acid complexes, after washing and drying of the complexes, can be eluted by means of an elution buffer.

Whatever the implementation mode, the solid phase-nucleic acid complexes thus obtained can be contacted with a mixture the components wherein are present for the purpose of amplifying the nucleic acid, either attached to said solid phase, or eluted therefrom.

Whatever the implementation mode, the incubation step can comprise maintaining the treated sample for 5 to 45 minutes, preferably for 15 to 35 minutes, and still more preferably for 25 minutes at a temperature lying between 45 and 85° C., preferably between 55 to 75° C., and still more preferably at 65° C.

According to the preceding implementation mode, after the incubation step, the sample can be returned to ambient temperature during at least several minutes, preferably 5 minutes.

“Multimeric structure” is understood to mean a polymer formed of repeated units of chemical or biological synthons. One example is cited in example 34.2 of the description in the patent application WO-A-02/090319. The skilled person is invited to refer to that document if he/she finds the information hereinafter expounded insufficient for his/her complete understanding on this subject. Many versions of such structures utilizable in the present invention are known, such as for example:
linear polymers (EP-A-0.561.722, EP-A-0.669.991),
branched polymers (WO-A-01/92361),
particles (EP-A-0 827 552),
dendrimers (U.S. Pat. No. 4,507,466; U.S. Pat. No. 4,568,737; U.S. Pat. No. 6,083,708),
polynucleotides, and
polypeptides.

If that proves necessary, the skilled person can also refer to these documents for perfect understanding on this subject.

“Detectable label” is understood to mean at least one label capable of directly generating a detectable signal. For example the presence of biotin is regarded as direct labeling, since it is detectable, even if it is possible subsequently to combine it with labeled streptavidin. A non-limitative list of these labels follows:
enzymes which produce a detectable signal for example by colorimetry, fluorescence or luminescence, such as horseradish peroxidase, alkaline phosphatase, β-galactosidase or glucose-6-phosphate dehydrogenase,
chromophores such as fluorescent, luminescent or colorant compounds,
groups of electron density detectable by electron microscopy or by an electrical property such as their conductivity, amperometry, voltametry or impedance,
detectable groups, for example those whose molecules are of sufficient size to cause detectable changes in their physical and/or chemical properties; this detection can be performed by optical methods such as diffraction, surface plasmon resonance, surface variation, variation in the contact angle or physical methods such as atomic force spectroscopy or the tunnel effect,
radioactive molecules such as ³²P, ³⁵S or ¹²⁵I.
   The label is preferably not a radioactive label, in order to avoid the safety problems associated with these labels.

In a particular implementation mode of the present invention the label is detectable electrochemically, and in particular the label is a derivative of an iron complex, such as a ferrocene.

The term “nucleic acid” signifies a chain of at least two desoxyribonucleotides or ribonucleotides possibly containing at least one modified nucleotide, for example at least one nucleotide containing a modified base such as inosine, methyl-5-desoxycytidine, dimethylamino-5-desoxyuridine, desoxyuridine, diamino-2,6-purine, bromo-5-desoxyuridine or any other modified base allowing hybridization. This polynucleotide can also be modified at the internucleotide linkage such as for example the phosphorothioates, the H-phosphonates, or the alkyl-phosphonates, at the skeleton such as for example the alpha-oligonucleotides (FR 2 607 507) or the PNA (M. Egholm et al., J. Am. Chem. Soc., 114, 1895-1897, 1992 or 2′ O-alkyl ribose and the LNA (BW, Sun et al., Biochemistry, 4160-4169, 43, 2004). The nucleic acid can be natural or synthetic, an oligonucleotide, a polynucleotide, a nucleic acid fragment, a ribosomal RNA, a messenger RNA, a transfer RNA, or a nucleic acid obtained by an enzymatic amplification technique such as:
PCR (Polymerase Chain Reaction), described in the U.S. Pat. No. 4,683,195, U.S. Pat. No. 4,683,202 and U.S. Pat. No. 4,800,159, and its derivative RT-PCR (Reverse Transcription PCR), particularly in a one step format, as described in the patent EP-B-0.569.272,
LCR (Ligase Chain Reaction), disclosed for example in the patent application EP-A-0.201.184,
RCR (Repair Chain Reaction), described in the patent application WO-A-90/01069,
3SR (Self Sustained Sequence Replication) with the patent application WO-A-90/06995,
NASBA (Nucleic Acid Sequence-Based Amplification) with the patent application WO-A-91/02818, and
TMA (Transcription Mediated Amplification) with the U.S. Pat. No. 5,399,491.
RCA (Rolling Circle Amplification) U.S. Pat. No. 6,576,448.
   The term amplicons is then used to refer to the nucleic acids generated by an enzymatic amplification technique.

Any of these modifications can be used in combination provided that at least one phosphate is present in the nucleic acid.

“Polypeptide” is understood to mean a chain of at least two amino acids.

“Amino acids” is understood to mean:
primary amino acids which code for proteins,
amino acids derived from enzymatic action, such as trans-4-hydroxyproline,
amino acids which are natural, but not present in proteins such as norvaline, N-methyl-L leucine, and staline (see Hunt S. in Chemistry and Biochemistry of the amino acids, Barett G. C., ed., Chapman and Hall, London, 1985), and
amino acids protected by chemical groups, utilizable in synthesis on solid support or in the liquid phase and non-natural amino acids.

“Purification step” is understood to mean in particular separation of the nucleic acids from microorganisms and from the cell components released in the lysis step that precedes the purification of the nucleic acids. These lysis steps are well known by way of example, lysis methods as described in the following patent applications can be used:
WO-A-00/60049 on lysis by sonication,
WO-A-00/05338 on combined magnetic and mechanical lysis,
WO-A-99/53304 on electrical lysis, and
WO-A-99/15621 on mechanical lysis.
   The skilled person will be able to use other well-known lysis methods such as thermal or osmotic shock or treatment with chaotropic agents, such as guanidium salts (U.S. Pat. No. 5,234,809).

This step normally makes it possible to concentrate the nucleic acids. By way of example, magnetic particles can be used (on this subject, see the U.S. Pat. No. 4,672,040 and U.S. Pat. No. 5,750,338), and the nucleic acids which have become attached to these magnetic particles can thus be purified by a washing step. This nucleic acid purification step is of particular interest if it is desired subsequently to amplify said nucleic acids. A particularly interesting implementation mode for these magnetic particles is described in the patent applications WO-A-97/45202 and WO-A-99/35500.

The term “solid support” as used here includes all materials onto which a nucleic acid can be attached. Synthetic materials or natural materials, which may be chemically modified, can be used as a solid support, in particular the polysaccharides such as materials based on cellulose, for example paper, derivatives of cellulose such as cellulose acetate and nitrocellulose, or dextran, polymers, copolymers, in particular those based on monomers of the styrene type, natural fibers such as cotton, and synthetic fibers such as nylon, inorganic materials such as silica, quartz, glasses and ceramics, latexes, magnetic particles, metal derivatives, gels, etc. The solid support can be in the form of a microtitration plate, a membrane, a particle or an essentially flat plate of glass or silicon or derivatives.

The appended examples represent particular implementation modes and cannot be regarded as limiting the scope of the present invention.

### EXAMPLE 1

### Labeling and Cleavage on Solid Phase of the PCR Amplification Product Followed by Analysis on an Affymetrix Chip

A—Myco 16 S PCR:

This experiment is performed on a model referred to as “Myco 16S”.

This name refers to amplicons generated by PCR from a sequence of 180 bases from a fragment of the gene coding for the 16S ribosomal RNA of Mycobacterium tuberculosis.

The conditions for the culturing and extraction of the mycobacteria and the amplification primers are given in the publication by Alain Troesch.: “Mycobacterium species identification and rifampin resistance testing with high-density DNA probe arrays” published in J. Clin. Microbiol. 1999, 37, pages 49 to 55.

The PCR is performed using preparations of genomic DNA (103 copies by PCR) as the starting template with the FastStart High Fidelity PCR System kit (Roche Diagnostic Corporation, Basel, Switzerland, Reference No.: 03 553 426 001) with 0.2 mM of each desoxyribonucleotide, 0.3 μM of primers and 0.4 μL of enzyme.

The PCR cycle parameters are as follows: 95° C. for 4 minutes then 35 cycles according to the following protocol: 95° C. for 30 seconds, then 55° C. for 30 seconds and finally 72° C. for 30 seconds. The mixture is then stored at 4° C. until the system stops.

The amplicons derived from the PCR described above are referred to below by the terms “16S PCR”.

B—Labeling in Homogenous Phase (Control):

Labeling in homogenous phase will be used as the reference technique with respect to the invention, and will be called the “control”.

A volume of 50 μL of 16S PCR diluted 1/5 in an amplification buffer provided in the kit is mixed with 75 μL of meta-biotin-phenylmethyldiazomethane, hereinafter referred to as “m-BioPMDAM”, (50 mM in dimethyl sulfoxide, hereinafter referred to as “DMSO”) and 102.5 μL H₂O, then incubated for 25 minutes at 95° C. 22.5 μL of 0.1 M HCl are then added to the reaction medium, and it is then again incubated at 95° C. for 5 minutes.

The reaction medium is then purified with the QIAQuick kit (QIAgen GmbH, Hilden, Germany. Reference No.: 28 306) using the supplier's protocol, then it is hybridized on a DNA chip (Affymetrix, Santa Clara, Calif.). The DNA chip used is designed for the analysis of the 213-415 region of the M20940 sequence (GenBank Reference No.) of the 16S DNA of Mycobacterium tuberculosis. This DNA chip is described, with the corresponding hybridization protocol, in the article by A. Troesch et al.: “Mycobacterium species identification and rifampin resistance testing with high-density DNA probe arrays” published in J. Clin. Microbiol. 1999, 37, pages 49 to 55.

C—Labeling and Cleavage in Heterogenous Phase:

In parallel, the PCR is treated according to the protocol described below.

A 10 μL volume of PCR (so as to use the same quantity of PCR as for the control described in paragraph B) is mixed with 25 μL of m-BioPMDAM (50 mM in DMSO), 5 μL of 0.2 M HCL, 140 μL of Isopropanol (final %=70% v/v), and 20 μL of MagPrep magnetic silica beads (Merck KGaA, Darmstadt, Germany. Reference No.: 1.01193.0001).

This mixture is incubated for 25 minutes at 65° C. then 5 minutes at ambient temperature. The magnetic residue is then washed three times with 250 μL of 70% isopropanol (30% v/v in H₂O), then the nucleic acids adsorbed on the silica are eluted in a mixture of 100 μL of EB buffer (Elution Buffer, QIAgen. Reference No.: 19086), to which are added 400 μL of hybridization buffer (6×SSPE (0.9M NaCl, 60 mM NaH₂PO₄, 6 mM EDTA, pH 7.4) and 0.05% (v/v) of Triton X-100). This buffer is prepared according to the protocol described in the publication by A. Troesch et al. described in the preceding paragraph, so as to reproduce the conditions used for the control.

D—Results and Conclusion:

The results in terms of percentage homology, signal intensity (I) and background noise (N) are assembled in Table 1 below:

TABLE 1
Comparative study of percentage homology, signal intensity (I) and
background noise (N) of a procedure using the invention
compared to a control not using it

| Experiment | % homology | I | N | I/N |
|---|---|---|---|---|
| Control | 99.3 | 3710 | 377 | 9.8 |
| Labeling and cleavage on | 99.3 | 4455 | 404 | 11.0 |
| solid phase (invention) | | | | |

In conclusion, the results obtained with a reaction procedure according to the invention are comparable to those obtained with a more “classical” procedure in two separate steps, purification and labeling. Moreover, the reaction procedure is much shorter, about 50% less time, in the case of labeling and cleavage on solid phase (say 15 minutes instead of half an hour).

It is also found that the intensity values measured are practically the same. This indicates that the purification yield is similar using the two purification methods (QIAquick or magnetic beads).

### EXAMPLE 2

### Labeling and Cleavage on Solid Phase in Presence of Label Molecule Bearing Two Biotin Groups

A—Labeling in Homogenous Phase (Control):

A 50 μL volume of 16S PCR diluted 1/10 in amplification buffer (this dilution is performed in order to test the sensitivity of the test, by reducing the concentration of amplicons) is mixed with 39.5 μL of 190 mM N,N′-bis(13-biotinoylamino-4,7,10-trioxa-tridecyl)-5-(diazomethyl)isophthalamide (hereinafter referred to as “bisBioPDAM”) in DMSO, 110.5 μL DMSO and 15 μL H₂O, then incubated for 25 minutes at 95° C. 35 μL of 0.1 M HCl is then added to the reaction medium, then it is again incubated for 5 minutes at 95° C.

The reaction medium is then purified with the QIAquick kit using the supplier's protocol, then it is hybridized on a DNA chip (Affymetrix, Santa Clara, Calif.) in the same manner as in Example 1.

B—Labeling and Cleavage in Heterogenous Phase

In parallel, the same PCR diluted 1/10 is treated according to another protocol. The 50 μL volume of PCR is mixed with 39.5 μL of 190 mM bisBioPDAM in DMSO, 5 μL of 0.4 M HCl, 175 μL of isopropanol (final %=70% v/v), 6.5 μL of DMSO, and a residue of 20 μL of MagPrep magnetic silica beads.

This mixture is incubated for 15 minutes at 80° C. then 5 minutes at ambient temperature. The magnetic residue is then washed three times with 250 μL of 70% isopropanol (30% v/v in H₂O), then the nucleic acids adsorbed on the silica are eluted in a mixture of 100 μL of EB buffer (see above), to which are added 400 μL of hybridization buffer. The hybridization is performed in the same manner as in Example 1.

C—Results and Conclusions:

The results in terms of percentage homology, signal intensity (I) and background noise (N) are assembled in Table 2 below:

TABLE 2
Comparative study of percentage homology, signal intensity (I) and
background noise (N) of a procedure using the invention
compared to a control not using it

| Experiment | % homology | I | N | I/N |
|---|---|---|---|---|
| Control | 99.1 | 1051 | 232 | 4.5 |
| Labeling and cleavage on | 99.1 | 4071 | 437 | 9.3 |
| solid phase (invention) | | | | |

In conclusion, the results obtained with a reaction procedure according to the invention are better than those obtained with a more “classical” procedure in two separate steps, purification and labeling, when bisBioPDAM is used, on a diluted PCR.

This molecule, which is more soluble than m-BioPMDAM, and bears two biotin groups, also makes it possible to improve the sensitivity of the test using labeling on solid support. This is visible in the improvement in the intensity values (I) and the signal to noise ratio (I/N) which are improved compared to the “classical” procedure.

The use of the reaction procedure and of bisBioPDAM thus makes it possible to obtain a robust test (high intensity value, low sensitivity to inter-test variations) for a lower copy number.

### EXAMPLE 3

### Labeling of PCR Amplification Products on Solid Phase Followed by Quantitative Analysis on an Agilent Chip

A—HBV PCR

In the text below, the term “HBV PCR” refers to the PCR amplification product of a 3200 base pair fragment from the genome of the hepatitis B virus, which is generated in the following manner.

Approximately 10³ copies of hepatitis (HBV) DNA, contained in a volume of 15 μL of supernatant from a cell culture, are amplified by PCR using the Expand High Fidelity kit (Roche Diagnostic corporation, Basel, Switzerland. Reference No.: 1 732 641).

For information, the final reaction medium used in the kit contains 1.5 mM of MgCl₂, 200 μM of each dNTP, and 2.6 units of a mixture of Taq and Pow DNA polymerases; 0.3 μM of primers P1 and P2 the sequences whereof are:

P1:
5′-CCGGAAAGCTTGAGCTCTTCTTTTTCACCTCTGCTAATCA-3′
P2:
5′-CCGGAAAGCTTGAGCTCTTCAAAAAGTTGCATGGTGCTGG-3′,

are added to the mixture, so as to enable the amplification of the expected 3200 nucleotide fragment.

A description of this PCR is given in the article by Gunther S. et al. “A novel method for efficient amplification of whole hepatitis B virus genome permits rapid functional analysis and reveals deletion mutants in immunosuppressed patients”, Journal of Virology, September 1995, pages 5437 to 5444.

This HBV PCR is used so as to have available a PCR acting on nucleic acid sequences much longer than with the myco model (3200 bases instead of 180 for myco 16S) in order to study the capture yield of the magnetic beads on two very different models.

B—Determination of Capture Yields:

A PCR amplification product of one part of the 16S gene of Mycoplasma tuberculosis, amplified in the same manner as described in Example 1, and in parallel an HBV PCR product, are treated in the following manner:

A 10 μL volume of PCR is mixed with 25 μL of m-BioPMDAM (50 mM in DMSO), 5 μL H₂O, 140 μL Isopropanol (final %=70% v/v), and 20 μL of MagPrep magnetic silica beads.

This mixture is incubated for 25 mins at 65° C. then 5 mins at ambient temperature. The magnetic residue is then washed three times with 250 μL of 70% isopropanol (30% v/v in H₂O), then the nucleic acids adsorbed on the silica are eluted in 50 μL of EB buffer.

In parallel, the same PCR solutions are labeled according to the same protocol (isopropanol replaced with H₂O) then purified according to the QIAquick protocol and eluted in a final volume of 50 μL.

The purification products and the initial PCRs are analyzed on a BioAnalyser 2100 (Agilent, Palo Alto, Calif., Unites States of America, Reference No.: G2940CA) using the supplier's protocol, in order to quantify them.

C—Results and Conclusion:

The values obtained are reproduced in Table 3 shown below:

TABLE 3
Comparison of the capture yield of nucleic acids in the
reaction medium by a classical procedure of the state
of the technology and a procedure according to the invention

| | Initial | QIAgen | Invention |
|---|---|---|---|
| HBV (ng/μL) | 8.2 | 3.6 | 7.7 |
| % Yield | | 44 | 94 |
| Myco (ng/μL) | 2.7 | 0.94 | 2.6 |
| % Yield | | 35 | 96 |

To conclude, these results together with those obtained on chips show that the reliberation of nucleic acids into the reaction medium is low. With regard to the QIAgen kit, the major part of the capture takes place on fragments of small size, which enables its utilization with hybridized cleavage products, hybridization on DNA chip in certain cases necessitating cleavage of the PCR products (see for example Zhang Y. et al. “Reproducible and inexpensive probe preparation for oligonucleotide arrays”, published in Nucleic Acids Res. 2001, 29, e66). The results presented in this example make it possible to show that the protocol used with the magnetic beads makes it possible to capture a wider fragment size range. This allows greater flexibility in the technology, since it is possible, from PCR products of very diverse size, to obtain, by modification of the cleavage, labeled DNA fragments of size suitable for the intended application, without any particular technical constraint.

### EXAMPLE 4

### Labeling and Cleavage of Genomic DNA on Solid Phase Followed by Analysis on an Affymetrix Chip

A—Preparation of the Genomic DNA:

The genomic DNA, which will be referred to below as “gDNA”, is extracted from an 18 hour liquid culture of Staphylococcus aureus in Trypticase soya medium (bioMérieux, Marcy, France. Reference No.: 41 146).

The gDNA is purified on a “Genomic Tips 500” ion exchange column (QIAgen. Reference No.: 10262) according to the supplier's protocol, and quantified by measurement of the absorbance at 260 nm.

B—Labeling and Hybridization of the gDNA:

A 10 μL volume of gDNA, containing about 10⁹ copies of bacterial genomes is mixed with 25 μL of m-BioPMDAM (50 mM in DMSO), 5 μL of sodium acetate pH=3, 140 μL of Isopropanol (final %=70% v/v), and 20 μL of MagPrep magnetic silica beads (Merck).

This mixture is incubated for 25 minutes at 65° C. then 5 minutes at ambient temperature. The magnetic residue is then washed three times with 250 μL of 70% isopropanol (30% v/v in H₂O), then the nucleic acids adsorbed on the silica are eluted in a mixture of 100 μL of EB buffer to which are added 400 μL of hybridization buffer (3M Tetra Methyl Ammonium Chloride, 10 mM Tris, pH 7.8, 0.01% Tween-20, 500 μg/mL Acetylated Bovine Serum Albumin, and 100 μg/mL Herring Sperm DNA. This buffer is prepared according to the protocol provided by Affymetrix in its user manual: CustomSeq resequencing Array protocol Version 2.0).

The mixture is hybridized on an Affymetrix chip designed for the analysis of the 16S gene, the characteristics and operating protocols for which are described in the article by G. Vernet et al. “Species differentiation and antibiotic susceptibility testing with DNA microarrays”, published in J. Appl. Microbiol., 2004, 96, pages 59 to 68 and C. Jay et al. “16S rRNA gene-based identification of Staphylococcus species using high density DNA probe array”, 10th international Symposium on staphylococci and Staphylococcal infections, Tsukuba, of October 2002.

C—Results and Conclusion:

The results in terms of percentage homology, signal intensity (I) and background noise (N) are assembled in Table 4 below:

TABLE 4
Percentage homology, signal intensity (I) and background noise (N)
results for a gDNA treatment procedure using the invention

| Experiment | % homology | I | N | I/N |
|---|---|---|---|---|
| Labeling and cleavage on | 92.9 | 2799 | 599 | 4.7 |
| solid phase (invention) | | | | |

In conclusion, this result is very satisfactory. The percentage homology of 93% makes it possible in an ideal manner to identify the bacterial species analyzed, and to differentiate it from the other species capable of being identified on the chip.

Knowing that it is possible to purify the gDNA on the same magnetic beads as those used for the purification of the labeled DNA, the whole of the protocol can be implemented (from the bacterial colony to the DNA ready to be hybridized) in one and the same tube, processed manually or in an automatic device.

Identical results from the point of view of the invention can be obtained by using other amplification techniques such as NASBA or TMA, which generate RNA amplicons directly.

1. A process for labeling and purification of nucleic acids of interest which are present in a biological sample to be treated, consisting in:
taking a single reaction vessel,
introducing into the reaction vessel:
the biological sample,
at least one labeling reagent for nucleic acids, said labeling reagent being stable to heat and of formula (0): 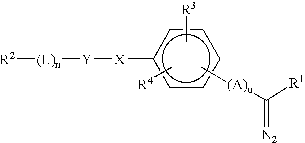
wherein:
R¹ represents H or an alkyl, aryl or substituted aryl group,
R² represents a detectable label or at least two detectable labels linked together by at least one multimeric structure,
L is a linking arm comprising a linear chain of at least two covalent bonds and n a whole number equal to 0 or 1,
R³ and R⁴ independently of one another represent: H, NO₂, Cl, Br, F, I, R²-(L)ₙ-Y—X—, OR, SR, NR₂, R, NHCOR, CONHR or COOR with R=alkyl or aryl,
A is a linking arm comprising at least one covalent double bond enabling the conjugation of the diazo group with the aromatic ring and u is a whole number lying between 0 and 2, preferably 0 or 1, and
Y—X— represents —CONH—, —NHCO—, —CH₂O— or —CH₂S—.
at least one solid support enabling the adsorption of said nucleic acids,
any ingredient necessary for the labeling of the nucleic acids and/or for the immobilization of said nucleic acids on the support,
incubating the contents of the reaction vessel, and
isolating the nucleic acids thus labeled.

2. The labeling and purification process as claimed in claim 1, wherein the nucleic acids treated consist of single-strand and/or double-strand, synthetic and/or natural DNA and/or RNA.

3. The labeling and purification process as claimed in claim 1, wherein the introduction of the labeling reagent enables:
the fragmentation of the nucleic acids in a non-specific manner to generate a plurality of nucleic acid fragments, and
the labeling of a plurality of these fragments at the terminal phosphate situated at the 3′ and/or 5′ end, said terminal phosphate having been liberated during the fragmentation.

4. The labeling and purification process as claimed in claim 3, wherein the labeling of the 3′ or 5′ end of a nucleic acid fragment is performed by attachment of a reactive group borne by a label onto the phosphate in the 2′ position, 3′ position or cyclic 2′-3′-mono-phosphate position, with reference to the ribose.

5. The labeling and purification process as claimed in claim 3, wherein the fragmentation and/or labeling of the 3′ or 5′ end of a nucleic acid fragment is performed by attachment of a nucleophilic, electrophilic or halide group borne by a label onto the phosphate in the 2′ position, 3′ position or cyclic 2′-3′-monophosphate position, with reference to the ribose.

6. The process as claimed in claim 3, wherein the fragmentation of the nucleic acids is performed by:
enzymatic means (nucleases),
chemical means (metal cations, such as Mg⁺⁺, Mn⁺⁺, Cu⁺⁺, Co⁺⁺ and/or Zn⁺⁺ ions, whether or not in combination with a chemical catalyst, for example N-methyl-imidazole, or any chemical molecule having an affinity for RNA and bearing an imidazole nucleus or a substituted analog) or
physical means (by sonication or by radiation).

7. The process as claimed in claim 3, wherein the labeling of the 3′ or 5′ end of a fragment of RNA is performed by attachment of a molecule R—X, where R consists of the label and X is the binding agent between the label and the RNA, such as a hydroxyl, amine, hydrazine, alkoxylamine, alkyl halide, phenylmethyl halide, iodoacetamide or maleimide group onto the phosphate linked to the 2′ position, 3′ position or the cyclic 2′-3′-monophosphate position of the ribose.

8. The process as claimed in claim 3, wherein the labeling on the phosphate group is effected by means of 5-(bromomethyl)-fluorescein.

9. The labeling and purification process as claimed in claim 1, wherein the labeling reagent is contacted with the nucleic acids in homogenous solution, in an essentially aqueous buffer, said labeling reagent being stable to heat and of formula (0).

10. The process as claimed in claim 9, wherein the labeling reagent used is of formula (1): 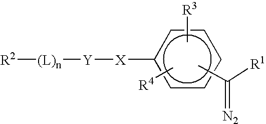
wherein:
R¹ represents H or an alkyl, aryl or substituted aryl group,
R² represents a detectable label or at least two detectable labels linked together by at least one multimeric structure,
L is a linking arm comprising a linear chain of at least two covalent bonds and n a whole number equal to 0 or 1,
R³ and R⁴ independently of one another represent: H, NO₂, Cl, Br, F, I, R²-(L)ₙ-Y—X—, OR, SR, NR₂, R, NHCOR, CONHR or COOR with R=alkyl or aryl, and
—Y—X— represents —CONH—, —NHCO—, —CH₂O— or —CH₂S—.

11. The process as claimed in claim 9, wherein the reagent is of formula (2): 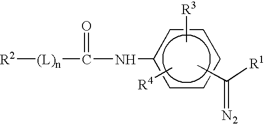
wherein:
R¹ represents H or an alkyl, aryl or substituted aryl group,
R² represents a detectable label or at least two detectable labels linked together by at least one multimeric structure,
L is a linking arm comprising a linear chain of at least two covalent bonds and n a whole number equal to 0 or 1, and
R³ and R⁴ independently of one another represent: H, NO₂, Cl, Br, F, I, R²-(L)ₙ-Y—X—, OR, SR, NR₂, R, NHCOR, CONHR or COOR with R=alkyl or aryl.

12. The process as claimed in claim 9, wherein the reagent is of formula (3): 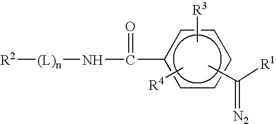
wherein:
R¹ represents H or an alkyl, aryl or substituted aryl group,
R² represents a detectable label or at least two detectable labels linked together by at least one multimeric structure,
L is a linking arm comprising a linear chain of at least two covalent bonds and n a whole number equal to 0 or 1, and
R³ and R⁴ independently of one another represent: H, NO₂, Cl, Br, F, I, R²-(L)ₙ-Y—X—, OR, SR, NR₂, R, NHCOR, CONHR or COOR with R=alkyl or aryl.

13. The process as claimed in claim 9, wherein the reagent is of formula (4): 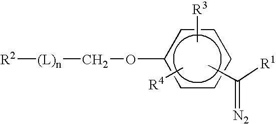
wherein:
R¹ represents H or an alkyl, aryl or substituted aryl group,
R² represents a detectable label or at least two detectable labels linked together by at least one multimeric structure,
L is a linking arm comprising a linear chain of at least two covalent bonds and n a whole number equal to 0 or 1, and
R³ and R⁴ independently of one another represent: H, NO₂, Cl, Br, F, I, R²-(L)ₙ-Y—X—, OR, SR, NR₂, R, NHCOR, CONHR or COOR with R=alkyl or aryl.

14. The process as claimed in claim 9, wherein the reagent is of formula (21): 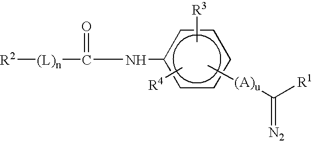
wherein:
R¹ represents H or an alkyl, aryl or substituted aryl group,
R² represents a detectable label or at least two detectable labels linked together by at least one multimeric structure,
L is a linking arm comprising a linear chain of at least two covalent bonds and n a whole number equal to 0 or 1,
A is a linking arm comprising at least one covalent double bond enabling the conjugation of the diazo group with the aromatic ring and u equals 1, and
R³ and R⁴ independently of one another represent: H, NO₂, Cl, Br, F, I, R²-(L)ₙ-Y—X—, OR, SR, NR₂, R, NHCOR, CONHR or COOR with R=alkyl or aryl.

15. The process as claimed in claim 9, wherein the reagent is of formula (22): 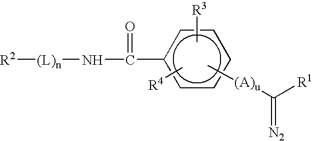
wherein:
R¹ represents H or an alkyl, aryl or substituted aryl group,
R² represents a detectable label or at least two detectable labels linked together by at least one multimeric structure,
L is a linking arm comprising a linear chain of at least two covalent bonds and n a whole number equal to 0 or 1,
A is a linking arm comprising at least one covalent double bond enabling the conjugation of the diazo group with the aromatic ring and u equals 1, and
R³ and R⁴ independently of one another represent: H, NO₂, Cl, Br, F, I, R²-(L)ₙ-Y—X—, OR, SR, NR₂, R, NHCOR, CONHR or COOR with R=alkyl or aryl.

16. The process as claimed in claim 9, wherein the reagent is of formula (23): 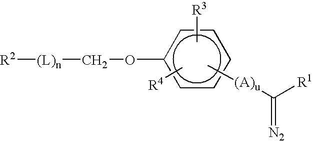
wherein:
R¹ represents H or an alkyl, aryl or substituted aryl group,
R² represents a detectable label or at least two detectable labels linked together by at least one multimeric structure,
L is a linking arm comprising a linear chain of at least two covalent bonds and n a whole number equal to 0 or 1,
A is a linking arm comprising at least one covalent double bond enabling the conjugation of the diazo group with the aromatic ring and u equals 1, and
R³ and R⁴ independently of one another represent: H, NO₂, Cl, Br, F, I, R²-(L)ₙ-Y—X—, OR, SR, NR₂, R, NHCOR, CONHR or COOR with R=alkyl or aryl.

17. The process as claimed in claim 9, wherein the labeling reagent used contains R³ and R⁴ which independently of one another represent: H, NO₂, OCH₃, —CO—NH—(CH₂)₃—(O—CH₂—CH₂)₃—CH₂—NH—R² or —CO—NH—(CH₂)₃—(O—CH₂—CH₂)₄—CH₂—NH—R².

18. The process as claimed in claim 9, wherein the reagent is of formula (7): 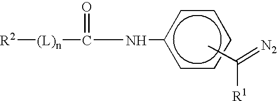
wherein:
R¹ represents H or an alkyl, aryl or substituted aryl group,
R² represents a detectable label or at least two detectable labels linked together by at least one multimeric structure, and
L is a linking arm comprising a linear chain of at least two covalent bonds and n a whole number equal to 0 or 1.

19. The process as claimed in claim 9, wherein the reagent is of formula (24): 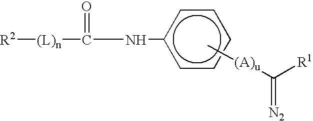
wherein:
R¹ represents H or an alkyl, aryl or substituted aryl group,
R² represents a detectable label or at least two detectable labels linked together by at least one multimeric structure,
L is a linking arm comprising a linear chain of at least two covalent bonds and n a whole number equal to 0 or 1, and
A is a linking arm comprising at least one covalent double bond enabling the conjugation of the diazo group with the aromatic ring and u equals 1.

20. The process as claimed in claim 9, wherein the structure R²-(L)ₙ- in the reagent is of formula (5): 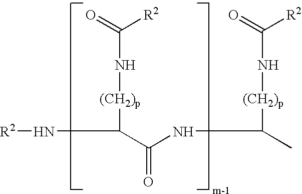
wherein:
R² represents a detectable label,
m is a whole number lying between 1 and 100, and
p is a whole number lying between 1 and 10.

21. A process for labeling and purification of nucleic acids of interest present in a biological sample to be treated, comprising:
taking a single reaction vessel,
introducing into the reaction vessel:
the biological sample,
at least one labeling reagent for nucleic acids, the labeling reagent being contacted with the nucleic acids in homogenous solution, in an essentially aqueous buffer, said labeling reagent being stable to heat, and of formula (6): 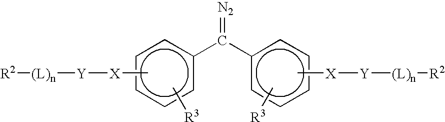
wherein:
R² represents a detectable label or at least two detectable labels linked together by at least one multimeric structure,
R³ represents H, NO₂, Cl, Br, F, I, R²-(L)ₙ-Y—X—, OR, SR, NR₂, R, NHCOR, CONHR or COOR with R=alkyl or aryl,
L is a linking arm comprising a linear chain of at least two covalent bonds and n a whole number equal to 0 or 1, and
—Y—X— represents —CONH—, —NHCO—, —CH₂O— or —CH₂S—,
at least one solid support enabling the adsorption of said nucleic acids,
any ingredient necessary for the labeling of the nucleic acids and/or for the immobilization of said nucleic acids on the support,
incubating the contents of the reaction vessel, and
isolating the nucleic acids thus labeled.

22. A process for labeling and purification of nucleic acids of interest present in a biological sample to be treated, comprising:
taking a single reaction vessel,
introducing into the reaction vessel:
the biological sample,
at least one labeling reagent for nucleic acids, the labeling reagent being contacted with the nucleic acids in homogenous solution, in an essentially aqueous buffer, said labeling reagent being stable to heat, and of formula (25): 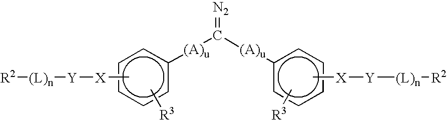
wherein:
R² represents a detectable label or at least two detectable labels linked together by at least one multimeric structure,
R³ represents H, NO₂, Cl, Br, F, I, R²-(L)ₙ-Y—X—, OR, SR, NR₂, R, NHCOR, CONHR or COOR with R=alkyl or aryl,
L is a linking arm comprising a linear chain of at least two covalent bonds and n a whole number equal to 0 or 1,
A is a linking arm comprising at least one covalent double bond enabling the conjugation of the diazo group with the aromatic ring and u equals 1, and
—Y—X— represents —CONH—, —NHCO—, —CH₂O— or —CH₂S—,
at least one solid support enabling the adsorption of said nucleic acids,
any ingredient necessary for the labeling of the nucleic acids and/or for the immobilization of said nucleic acids on the support,
incubating the contents of the reaction vessel, and
isolating the nucleic acids thus labeled.

23. The process as claimed in claim 9, wherein the reagent is of formula (14): 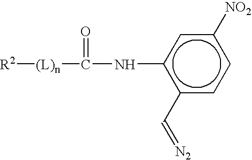
wherein:
R² represents a detectable label or at least two detectable labels linked together by at least one multimeric structure, and
L is a linking arm comprising a linear chain of at least two covalent bonds and n a whole number equal to 0 or 1.

24. The process as claimed in claim 9, wherein the reagent is of formula (26): 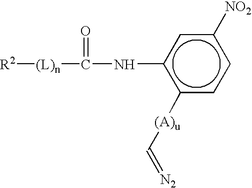
wherein:
R² represents a detectable label or at least two detectable labels linked together by at least one multimeric structure,
A is a linking arm comprising at least one covalent double bond enabling the conjugation of the diazo group with the aromatic ring and u equals 1, and
L is a linking arm comprising a linear chain of at least two covalent bonds and n a whole number equal to 0 or 1.

25. The process as claimed in claim 9, wherein the reagent is of formula (15): 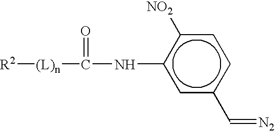
wherein:
R² represents a detectable label or at least two detectable labels linked together by at least one multimeric structure, and
L is a linking arm comprising a linear chain of at least two covalent bonds and n a whole number equal to 0 or 1.

26. The process as claimed in claim 9, wherein the reagent is of formula (27): 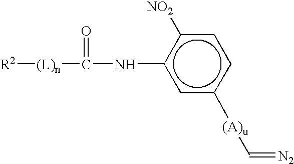
wherein:
R² represents a detectable label or at least two detectable labels linked together by at least one multimeric structure,
A is a linking arm comprising at least one covalent double bond enabling the conjugation of the diazo group with the aromatic ring and u equals 1, and
L is a linking arm comprising a linear chain of at least two covalent bonds and n a whole number equal to 0 or 1.

27. The process as claimed in claim 9 wherein the constituent L of the reagent contains a —(O—CH₂—CH₂)— moiety, repeated from 1 to 20 times.

28. A process for labeling and purification of nucleic acids of interest present in a biological sample to be treated, comprising:
taking a single reaction vessel,
introducing into the reaction vessel:
the biological sample,
at least one labeling reagent for nucleic acids, the labeling reagent enabling the labeling and the fragmentation a single or double-strand nucleic acid according to the following steps:
fragmenting the nucleic acid,
attaching a label onto at least one of the fragments by means of a labeling reagent selected from the compounds of formula (19): 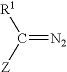
wherein:
R¹ represents H or an alkyl, aryl or substituted aryl group, and
Z contains a detectable label,
said reagent coupling covalently and mainly onto at least one phosphate of said fragment,
at least one solid support enabling the adsorption of said nucleic acids,
any ingredient necessary for the labeling of the nucleic acids and/or for the immobilization of said nucleic acids on the support,
incubating the contents of the reaction vessel, and
isolating the nucleic acids thus labeled.

29. The process as claimed in claim 28, wherein the labeling reagent is selected from the compounds of formula (20): 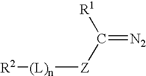
wherein:
R¹ represents H or an alkyl, aryl or substituted aryl group,
R² is a detectable label or at least two detectable labels linked together by at least one multimeric structure,
L is a linking arm comprising a linear chain of at least two covalent bonds and n is equal to 0 or 1, and
Z is selected from: 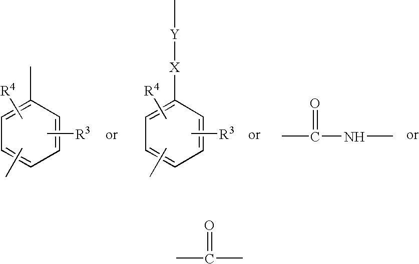
wherein:
R³ and R⁴ independently of one another represent: H, NO₂, Cl, Br, F, I, R²-(L)ₙ-Y—X—, OR, SR, NR₂, R, NHCOR, CONHR or COOR with R=alkyl or aryl, and
—Y—X— represents —CONH—, —NHCO—, —CH₂O— or —CH₂S—.

30. The process as claimed in claim 28, wherein Z is:

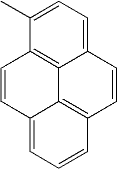

31. The process as claimed in claim 28, wherein the fragmentation and the labeling are performed in two steps.

32. The process as claimed in claim 28, wherein the fragmentation and the labeling are performed in one step.

33. The process as claimed in claim 28, wherein the labeling is performed in essentially aqueous homogeneous solution.

34. The process as claimed in claim 28, wherein the fragmentation is performed by enzymatic, physical or chemical means.

35. A process for labeling and purification of nucleic acids of interest present in a biological sample to be treated, comprising:
taking a single reaction vessel,
introducing into the reaction vessel:
the biological sample,
at least one labeling reagent for nucleic acids, the labeling reagent being contacted in homogeneous solution, in an essentially aqueous buffer, said labeling reagent being stable to heat and of formula (8): 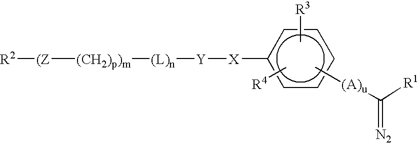
wherein:
R¹ represents H or an alkyl, aryl or substituted aryl group,
R² represents a detectable label or at least two detectable labels linked together by at least one multimeric structure,
L is a linking arm comprising a linear chain of at least two covalent bonds and n a whole number equal to 0 or 1,
R³ and R⁴ independently of one another represent: H, NO₂, Cl, Br, F, I, R²-(L)ₙ-Y—X—, OR, SR, NR₂, R, NHCOR, CONHR, COOR, —CO—NH—(CH₂)₃—(O—CH₂—CH₂)₃—CH₂—NH—R² or —CO—NH—(CH₂)₃—(O—CH₂—CH₂)₄—CH₂—NH—R² with R=alkyl or aryl,
A is a linking arm comprising at least one covalent double bond enabling the conjugation of the diazo group with the aromatic ring and u is a whole number lying between 0 and 2, preferably 0 or 1,
—Y—X— represents —CONH—, —NHCO—, —CH₂O— or —CH₂S—,
-Z- represents —NH—, —NHCO—, —CONH— or —O—,
m is a whole number lying between 1 and 10, and
p is a whole number lying between 1 and 10,
at least one solid support enabling the adsorption of said nucleic acids,
any ingredient necessary for the labeling of the nucleic acids and/or for the immobilization of said nucleic acids on the support,
incubating the contents of the reaction vessel, and
isolating the nucleic acids thus labeled.

36. The process as claimed in claim 35, wherein the reagent is of formula (9): 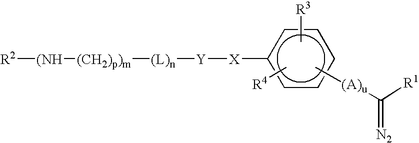
wherein:
R¹ represents H or an alkyl, aryl or substituted aryl group,
R² represents a detectable label or at least two detectable labels linked together by at least one multimeric structure,
L is a linking arm comprising a linear chain of at least two covalent bonds and n a whole number equal to 0 or 1,
R³ and R⁴ independently of one another represent: H, NO₂, Cl, Br, F, I, R²-(L)ₙ-Y—X—, OR, SR, NR₂, R, NHCOR, CONHR, COOR, —CO—NH—(CH₂)₃—(O—CH₂—CH₂)₃—CH₂—NH—R² or —CO—NH—(CH₂)₃—(O—CH₂—CH₂)₄—CH₂—NH—R² with R=alkyl or aryl,
A is a linking arm comprising at least one covalent double bond enabling the conjugation of the diazo group with the aromatic ring and u is a whole number lying between 0 and 2, preferably 0 or 1,
—Y—X— represents —CONH—, —NHCO—, —CH₂O— or —CH₂S—,
m is a whole number lying between 1 and 10, and
p is a whole number lying between 1 and 10.

37. The process as claimed in claim 35, wherein, in the formula of the reagent, p is less than or equal to m.

38. The process as claimed in claim 35, wherein the reagent is of formula (10): 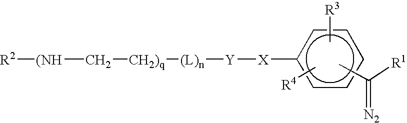
wherein:
R¹ represents H or an alkyl, aryl or substituted aryl group,
R² represents a detectable label or at least two detectable labels linked together by at least one multimeric structure,
L is a linking arm comprising a linear chain of at least two covalent bonds and n a whole number equal to 0 or 1,
R³ and R⁴ independently of one another represent: H, NO₂, Cl, Br, F, I, R²-(L)ₙ-Y—X—, OR, SR, NR₂, R, NHCOR, CONHR, COOR, —CO—NH—(CH₂)₃—(O—CH₂—CH₂)₃—CH₂—NH—R² or —CO—NH—(CH₂)₃—(O—CH₂—CH₂)₄—CH₂—NH—R² with R=alkyl or aryl, and
q is a whole number lying between 1 and 10.

39. The process as claimed in claim 35, wherein the reagent is of formula (11): 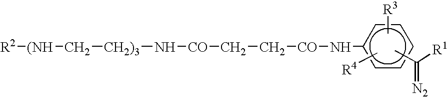
wherein:
R¹ represents H or an alkyl, aryl or substituted aryl group,
R² represents a detectable label or at least two detectable labels linked together by at least one multimeric structure,
L is a linking arm comprising a linear chain of at least two covalent bonds and n a whole number equal to 0 or 1, and
R³ and R⁴ independently of one another represent: H, NO₂, Cl, Br, F, I, R²-(L)ₙ-Y—X—, OR, SR, NR₂, R, NHCOR, CONHR, COOR, —CO—NH—(CH₂)₃—(O—CH₂—CH₂)₃—CH₂—NH—R², or —CO—NH—(CH₂)₃—(O—CH₂—CH₂)₄—CH₂—NH—R² with R=alkyl or aryl.

40. The process as claimed in claim 35, wherein in the reagent, R² comprises a D-Biotin residue of formula (12):

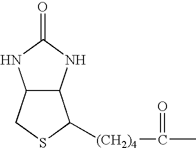

41. The process as claimed in any claim 35, wherein in the reagent, R¹ comprises: CH₃, and R³ and R⁴ each represent: H.

42. The process as claimed in claim 35, wherein the structure -(L)ₙ- of the reagent comprises:
spermine or N,N′-bis(3-aminopropyl)-1,4-diaminobutane: NH₂—(CH₂)₃—NH—(CH₂)₄—NH—(CH₂)₃—NH₂, or
spermidine or N-(3-aminopropyl)-1,4-butanediamine: H₂N—(CH₂)₄—NH—(CH₂)₃—NH₂, or
a derivative containing an alanine moiety: NH₂—CH₂—CH₂—COOH.

43. A process for labeling and purification of nucleic acids of interest present in a biological sample to be treated, comprising:
taking a single reaction vessel,
introducing into the reaction vessel:
the biological sample,
at least one labeling reagent for nucleic acids, the heat-stable labeling reagent is contacted in homogenous solution, in an aqueous buffer, and is of formula (13): 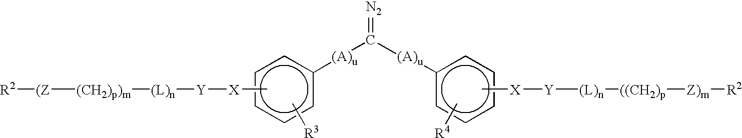
wherein:
R¹ represents H or an alkyl, aryl or substituted aryl group,
R² represents a detectable label or at least two detectable labels linked together by at least one multimeric structure,
L is a linking arm comprising a linear chain of at least two covalent bonds and n a whole number equal to 0 or 1,
R³ and R⁴ independently of one another represent: H, NO₂, Cl, Br, F, I, R²-(L)ₙ-Y—X—, OR, SR, NR₂, R, NHCOR, CONHR, COOR, —CO—NH—(CH₂)₃—(O—CH₂—CH₂)₃—CH₂—NH—R² or —CO—NH—(CH₂)₃—(O—CH₂—CH₂)₄—CH₂—NH—R² with R=alkyl or aryl,
A is a linking arm comprising at least one covalent double bond enabling the conjugation of the diazo group with the aromatic ring and u is a whole number lying between 0 and 2, preferably 0 or 1,
—Y—X— represents —CONH—, —NHCO—, —CH₂O— or —CH₂S—,
-Z- represents —NH—, —NHCO—, —CONH— or —O—,
m is a whole number lying between 1 and 10, and
p is a whole number lying between 1 and 10,
at least one solid support enabling the adsorption of said nucleic acids,
any ingredient necessary for the labeling of the nucleic acids and/or for the immobilization of said nucleic acids on the support,
incubating the contents of the reaction vessel, and
isolating the nucleic acids thus labeled.

44. The process as claimed in claim 43, wherein the reagent is of formula (16): 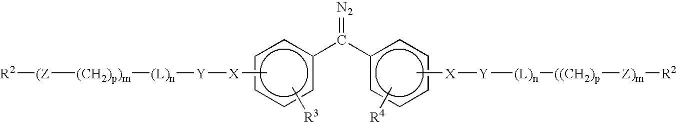
wherein:
R¹ represents H or an alkyl, aryl or substituted aryl group,
R² represents a detectable label or at least two detectable labels linked together by at least one multimeric structure,
L is a linking arm comprising a linear chain of at least two covalent bonds and n a whole number equal to 0 or 1,
R³ and R⁴ independently of one another represent: H, NO₂, Cl, Br, F, I, R²-(L)ₙ-Y—X—, OR, SR, NR₂, R, NHCOR, CONHR, COOR, —CO—NH—(CH₂)₃—(O—CH₂—CH₂)₃—CH₂—NH—R², or —CO—NH—(CH₂)₃—(O—CH₂—CH₂)₄—CH₂—NH—R² with R=alkyl or aryl,
—Y—X— represents —CONH—, —NHCO—, —CH₂O— or —CH₂S—,
-Z- represents —NH—, —NHCO—, —CONH— or —O—,
m is a whole number lying between 1 and 10, and
p is a whole number lying between 1 and 10.

45. The process as claimed in claim 35 wherein the constituent L of the reagent contains an —(O—CH₂—CH₂)— moiety, repeated from 1 to 20 times, -Z- then being represented by —NH—, —NHCO— or —CONH—.

46. The process as claimed in claim 1, wherein the solid support consists of particles of silica.

47. The process as claimed in claim 1, wherein the solid support consists of magnetic particles covered with silica.

48. The process as claimed in claim 46 wherein the particles of silica comprising the solid support have particle sizes lying between 0.1 and 500 μm.

49. The process as claimed in claim 1, wherein one of the supplementary ingredients enabling the labeling consists of an alcohol preferably Isopropanol.

50. The process as claimed in claim 49, wherein Isopropanol comprises 70% v/v of the final mixture.

51. The process as claimed in claim 1, wherein one of the supplementary ingredients enabling the cell liberation and thus the adsorption of the nucleic acids onto the solid support consists of a chaotropic agent.

52. The process as claimed in claim 51, wherein the chaotropic agent used is a guanidium salt, sodium iodide, potassium iodide, sodium (iso)thiocyanate, urea or mixtures of these derivatives.

53. The process as claimed in claim 52, wherein the guanidium salt used is guanidium (iso)thiocyanate.

54. The process as claimed in claim 1, wherein the solid phase-nucleic acid complexes are separated from the liquids by sedimentation and rejection of the supernatant, then washing of the complexes with a washing buffer containing a chaotropic substance.

55. The process as claimed in claim 54, wherein the solid phase-nucleic acid complexes, after washing with the washing buffer, are then washed again with one or several organic solvents, and then subjected to a drying process.

56. The process as claimed in claim 55, wherein the nucleic acid present in the solid phase-nucleic acid complexes, after washing and drying of the complexes, is eluted by means of an elution buffer.

57. The process as claimed in claim 1, wherein the solid phase-nucleic acid complexes thus obtained are contacted with a mixture wherein the components are present for the purpose of amplifying the nucleic acid, whether attached to said solid phase, or eluted therefrom.

58. The process as claimed in claim 1, wherein the incubation step comprises maintaining the treated sample for 5 to 45 minutes, at a temperature lying between 45 and 85° C.

59. The process as claimed in claim 58, wherein after the incubation step, the sample is brought back to ambient temperature for approximately 5 minutes.

60. The labeling and purification process as claimed in claim 21, wherein the nucleic acids treated consist of single-strand and/or double-strand, synthetic and/or natural DNA and/or RNA.


SEQUENCE LISTING (2 sequences)

SEQ ID NO: 1 - DNA, 40 bp, hepatitis B virus
ccggaaagcttgagctcttctttttcacctctgctaatca

SEQ ID NO: 2 - DNA, 40 bp, hepatitis B virus
ccggaaagcttgagctcttcaaaaagttgcatggtgctgg